(19) United States
(12) Patent Application Publication
(10) Pub. No.: US 2007/0248494 A1
(43) Pub. Date: Oct. 25, 2007

(54) Multi-Mode Modular Method and Apparatus for Micro-titer Plate Analysis

(76) Inventors: Michael F. Mokelke, Portola Valley, CA (US); Andriy Ponomaryov, Santa Clara, CA (US); Brian Quast, San Jose, CA (US); Pavel Vodkin, Sunnyvale, CA (US)

Correspondence Address: GLENN PATENT GROUP, 3475 EDISON WAY, SUITE L, MENLO PARK, CA, 94025, US

(21) Appl. No.: 11/739,074

(22) Filed: Apr. 23, 2007

Related U.S. Application Data
(60) Provisional application No. 60/745,504, filed on Apr. 24, 2006.

(51) Int. Cl. G01N 21/64; C12M 1/34; G01N 21/66

(52) U.S. Cl. 422/82.08; 435/288.7

(57) ABSTRACT

A reconfigurable microplate reader comprises a plurality of user installable detection modules. Each module comprises a self-contained detector for a microplate reader. Each module reconfigures the microplate reader to implement at least one specific detection scheme for detecting any of luminescence, fluorescence, and absorbance and/or reconfiguring said microplate reader to implement any of a fluorometer and a luminometer. A microplate reader platform has a port for coupling at least one user installable detection module thereto. The platform also comprises a user interface from the platform to the module, a machine interface from the platform to the module, and a platform configuration mechanism for recognizing a specific detection scheme for an installed detection module and for configuring the platform to support the specific detection scheme.

### CROSS REFERENCE TO RELATED APPLICATIONS

This application claims priority to U.S. provisional patent application Ser. No. 60/745,504, filed Apr. 24, 2006, which application is incorporated herein in its entirety by this reference thereto.

### BACKGROUND OF THE INVENTION

1. Technical Field

The invention relates to micro-titer plate analysis. More particularly, the present invention relates to a multi-mode modular method and apparatus for micro-titer analysis.

2. Description of the Prior Art

Science is an inherently competitive endeavor. Resources, such as time, equipment budget, lab space, etc. are universally valuable. Scientists in academic, government, and industrial research labs need to perform their research in a rapid and cost effective manner. This may require performing a wide variety of biological and biochemical assays and then analyzing the data acquired from these assays, ideally, in a cost effective, efficient manner in a user friendly environment.

Biological assays of greatest importance typically use one or more of the following basic detection modalities:
Florescence;
Luminescence; or
Absorbance.

In addition, certain assays may require the combination, modification, or permutation of the three basic detection modalities into modalities, such as those that include time resolved fluorescence, fluorescence lifetime, fluorescence polarization, fluorescence resonance energy transfer, and/or assays using multiple excitation and emission wavelengths.

Biological assays are typically performed in arrays of sample chambers or wells in micro-titer plates or microplates to increase throughput, automation, and ease of use, as well as to provide an optimal interface with standard laboratory infrastructure. Microplate formats typically comprise multiples of 8 or 12 wells, such as 8, 12, 24, 48, 96, 384, 1536, etc.

Commercially available microplate readers can accommodate many of the microplate formats and detection modality requirements described above. However, some scientists are unable or unwilling to purchase larger and more expensive multimode microplate readers or more than one single modality microplate reader to satisfy their multimode detection requirements. The reasons for this include, but are not limited to, lack of money, lack of lab space, and resistance to having multiple microplate readers, each with its own need for training in operation and software, maintenance and upgrading, spare parts, etc.

It would be desirable to have a microplate reader that could be inexpensively and simply upgraded or reconfigured by the user from an initial configuration having an initial set of detection modalities and other features, as received from the factory, to a new configuration having a second set of detection modalities and other features, so as to accommodate a different set of biological and biochemical assays without requiring a field service visit or returning the instrument to the factory for upgrading.

It would also be desirable if such a microplate reader was available also having a relatively small footprint, low initial cost, and which could be upgraded inexpensively by its user to meet a wide variety of future detection modality needs.

It would also be desirable if such a microplate reader was available that also provided a flexible interface for external or internal control or programming, data transfer, e.g. using transportable memory devices, data analysis, and functional enhancements, such as the addition of bar code readers, RF ID tags, etc., Ethernet, and/or other network interfaces.

### SUMMARY OF THE INVENTION

The invention provides a microplate reader that is inexpensively and simply upgraded or reconfigured by the user from an initial configuration having an initial set of detection modalities and other features, as received from the factory, to a new configuration having a second set of detection modalities and other features, so as to accommodate a different set of biological and biochemical assays without requiring a field service visit or returning the instrument to the factory for upgrading.

The also invention provides a microplate reader that has a relatively small footprint, low initial cost, and which is upgradable inexpensively by its user to meet a wide variety of future detection modality needs.

The also invention provides a microplate reader that provides a flexible interface for external or internal control or programming, data transfer, e.g. using transportable memory devices, data analysis, and functional enhancements, such as the addition of bar code readers, RF ID tags, etc., Ethernet, and/or other network interfaces.

To this end, the presently preferred embodiment of the invention is a reconfigurable microplate reader that comprises a base with a transport mechanism and facilities for a user interface, and a plurality of user installable detection modules. Each module comprises a self-contained detector for a microplate reader. Each module reconfigures the microplate reader to implement at least one specific detection scheme for detecting any of luminescence, fluorescence, and absorbance and/or reconfiguring said microplate reader to implement any spectroscopy detection technologies. Using dedicated sensors and electronics for each mode of detection allows optimization of the module design for particular application, and further improves metrological parameters of the device such as signal/noise precision and dynamic range.

The invention also comprises a microplate reader platform having a port for coupling at least one user installable detection module thereto. The platform also comprises a user interface from the platform to the module, a machine interface from the platform to the module, and a platform configuration mechanism for recognizing a specific detection scheme for an installed detection module and for configuring the platform to support the specific detection scheme.

### BRIEF DESCRIPTION OF THE DRAWINGS

FIG. 1 is a perspective view of one embodiment of a multi-mode modular apparatus for micro-titer analysis according to the invention;

FIG. 2 is a plan view of a multi-mode modular apparatus for micro-titer analysis according to the invention.

FIG. 3 is a high level block diagram of a multi-mode modular apparatus for micro-titer analysis according to the invention;

FIG. 4 is a more detailed block diagram of a multi-mode modular apparatus for micro-titer analysis according to the invention;

FIG. 5 is a block schematic diagram of a multi-mode modular apparatus for micro-titer analysis according to the invention;

FIG. 6 is a block diagram showing one of the possible embodiments of microplate movement in a multi-mode modular apparatus for micro-titer analysis according to the invention;

FIG. 7 is a high level block diagram showing a fluorescence module in a multi-mode modular apparatus for micro-titer analysis according to the invention;

FIG. 8 is a more detailed block diagram showing a fluorescence module in a multi-mode modular apparatus for micro-titer analysis according to the invention;

FIG. 9 is a schematic diagram showing a fluorescence module in a multi-mode modular apparatus for micro-titer analysis according to the invention;

FIG. 10 is a block diagram showing a fluorescence module optical system in a multi-mode modular apparatus for micro-titer analysis according to the invention;

FIG. 11 is a graph showing luminescence sensitivity in a multi-mode modular apparatus for micro-titer analysis according to the invention;

FIG. 12 is a block diagram showing an absorbance module in a multi-mode modular apparatus for micro-titer analysis according to the invention;

FIG. 13 is a screen shot of a user interface in a multi-mode modular apparatus for micro-titer analysis according to the invention;

FIG. 14 is a screen shot of a user interface read screen in a multi-mode modular apparatus for micro-titer analysis according to the invention;

FIG. 15 is a screen shot of a user interface read screen showing setup and modification of protocols in a multi-mode modular apparatus for micro-titer analysis according to the invention;

FIG. 16 is a screen shot of a user interface results screen in a multi-mode modular apparatus for micro-titer analysis according to the invention; and

FIG. 17 is a screen shot of a user interface read screen showing additional data handling in a multi-mode modular apparatus for micro-titer analysis according to the invention.

### DETAILED DESCRIPTION OF THE INVENTION

The invention provides a microplate reader that is inexpensively and simply upgraded or reconfigured by the user from an initial configuration having an initial set of detection modalities and other features, as received from the factory, to a new configuration having a second set of detection modalities and other features, so as to accommodate a different set of biological and biochemical assays without requiring a field service visit or returning the instrument to the factory for upgrading.

The also invention provides a microplate reader that has a relatively small footprint, low initial cost, and which is ugradable inexpensively by its user to meet a wide variety of future detection modality needs.

The also invention provides a microplate reader that provides a flexible interface for external or internal control or programming, data transfer, e.g. using transportable memory devices, data analysis, and functional enhancements, such as the addition of bar code readers, RF ID tags, etc., Ethernet, and/or other network interfaces.

To this end, the presently preferred embodiment of the invention is a reconfigurable microplate reader that comprises a plurality of user installable detection modules. Each module comprises a self-contained detector for a microplate reader. Each module reconfigures the microplate reader to implement at least one specific detection scheme for detecting any of luminescence, fluorescence, and absorbance and/or reconfiguring said microplate reader to implement any spectroscopy detection technologies. Using dedicated sensors and electronics for each mode of detection allows optimization of the module design for particular application, and further improves metrological parameters of the device such as signal/noise precision and dynamic range.

The invention also comprises a microplate reader platform having a port for coupling at least one user installable detection module thereto. The platform also comprises a user interface from the platform to the module, a machine interface from the platform to the module, and a platform configuration mechanism for recognizing a specific detection scheme for an installed detection module and for configuring the platform to support the specific detection scheme.

Features of the invention include a low initial cost and footprint and high flexibility. The invention provides multiple initial assay modalities and a reconfigurable architecture that enables rapid changing of assay modalities by the user. Features also include flexible software and interfacing capabilities and high ease of use and user friendliness. A user interface includes a touch screen display/user interface. The invention offers user upgradeability and provides data transportability via memory devices, such as a USB drive.

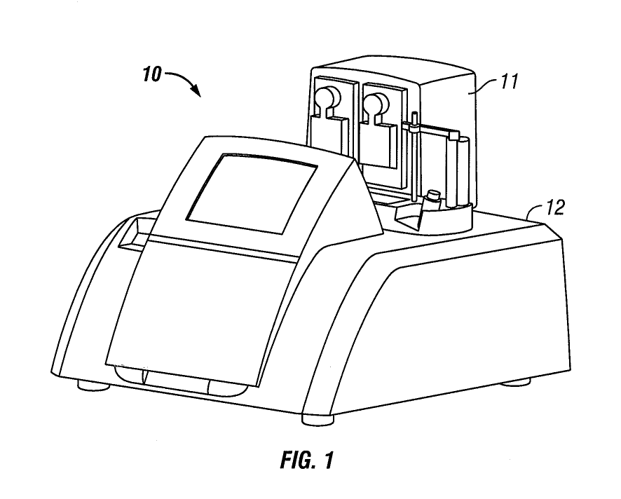

FIG. 1 is a perspective view of a multi-mode modular apparatus 10 for micro-titer analysis according to the invention. The invention provides a platform 12 that is adapted to receive a user installable module 11. Thus, the preferred embodiment of the invention comprises a multimode microplate reader platform, as shown in FIG. 1, that is adapted to receive modules that configure the reader to function as a luminescence detector in either of two or more modes, a fluorescence detector, an absorbance detector, a dedicated fluorometer, or a dedicated luminometer.

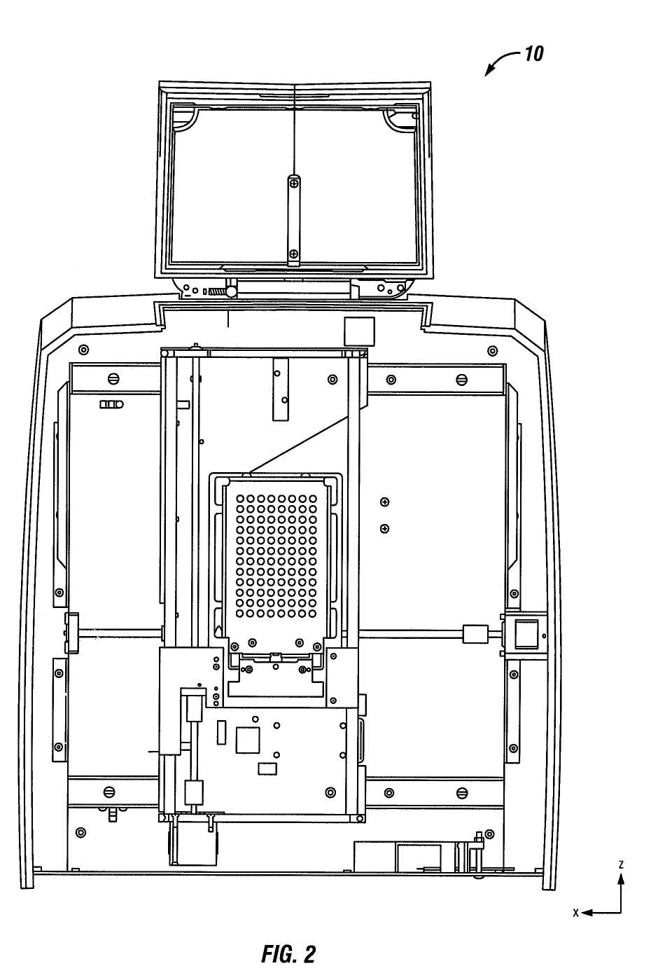

FIG. 2 is a plan view of a multi-mode modular apparatus for micro-titer analysis according to the invention.

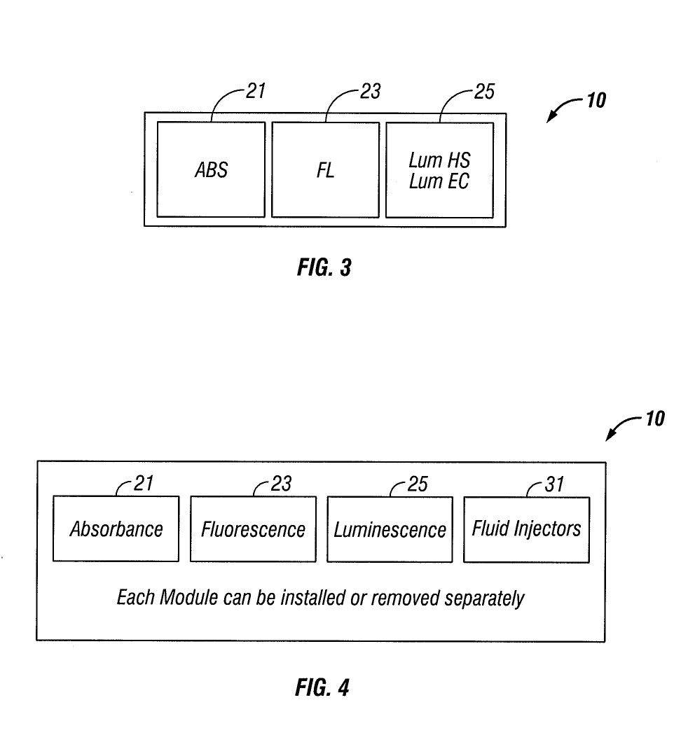

FIG. 3 is a high level block diagram of a multi-mode modular apparatus for micro-titer analysis according to the invention. The preferred embodiment comprises a plurality of dedicated channels for the detection of different parameters, where each detection technology has a specific position on a rack within the platform. These channels are referred to herein as modules, as discussed above in connection with FIG. 1. For example, fluorescence detection has a position in the platform for a fluorescent module FL 23. In the presently preferred embodiment, there are three specific positions: absorbance ABS 21, fluorescence FL 23, and luminescence high sensitivity LumHS (High Sensitivity PMT Detector)/luminescence assay specific LumEC (Photodiode Detector) 25. Those skilled in the art will appreciate that other configurations are possible within the scope of the invention.

FIG. 4 is a more detailed block diagram of a multi-mode modular apparatus for micro-titer analysis according to the invention. Each module can be installed or removed separately. Each channel is dedicated, thus allowing it to be optimized for the best detection limits. The ABS and FL modules can be factory installed or user installed. The LumHS module is typically factory installed, but could also be user installed. The LumEC module can be user installed. The channels can be electrically defined by a series of pins in a connector within a single bay in the platform, where the platform is configurable to support a single detector at any given time. Alternatively, a separate bay may be provided that is dedicated for each detector. In FIG. 4, a channel is shown for a plurality of fluid injectors 31. Other input and output functions may likewise be added tot he platform via various channels.

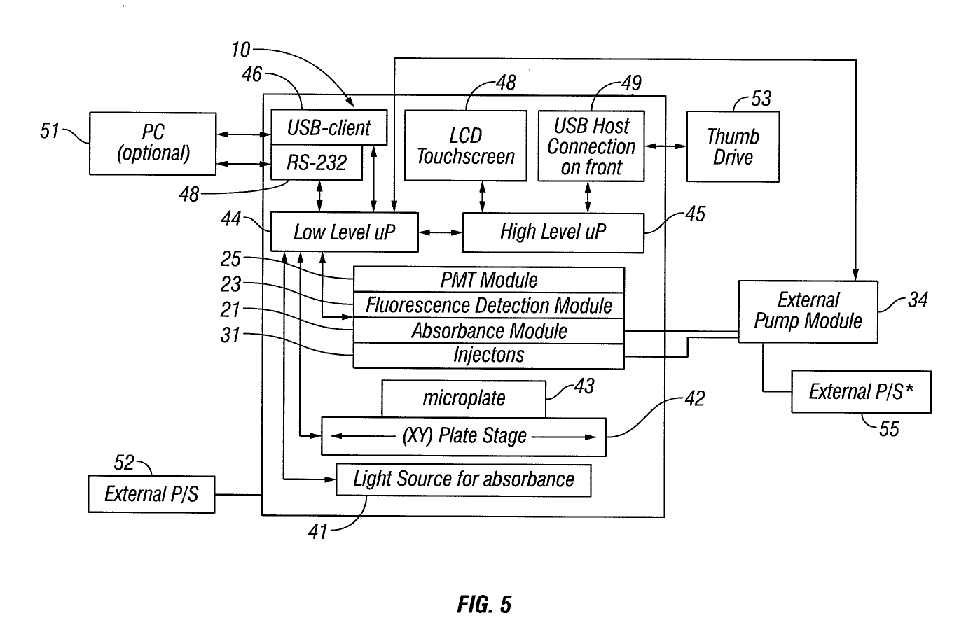

FIG. 5 is a block schematic diagram of a multi-mode modular apparatus for micro-titer analysis according to the invention. In FIG. 5, the platform 10 comprises a channel for an absorbance module 21, a fluorescence detection module 23, a luminescence detection module PMT 25, and fluid injectors 31, as discussed above in connection with FIG. 4. The platform provides a light source 41 for use in absorbance detection and an XY plate stage 42 for effecting XY movement of a microplate 43. An external pump module 54 and external power source 55 for the pump module is coupled to the platform for use when the reader is configured for absorbance detection. The modules are controlled by a low level microprocessor 44 and the overall platform is controlled by a high level microprocessor 45. Those skilled in the art will appreciate that the system architecture may include one or more processors and memory therefor. The high level microprocessor support the user interface, which in this embodiment comprises an LCD touch screen 48 and user accessible USB connector 49, which is located on the front of the reader in this embodiment, and which allows a user to load and store data to and from the reader with an external USB thumb drive 53. The reader also provides an RS-232 interface 47 and USB client 46 for connection to an external PC 51. The reader is powered by an external power supply 52.

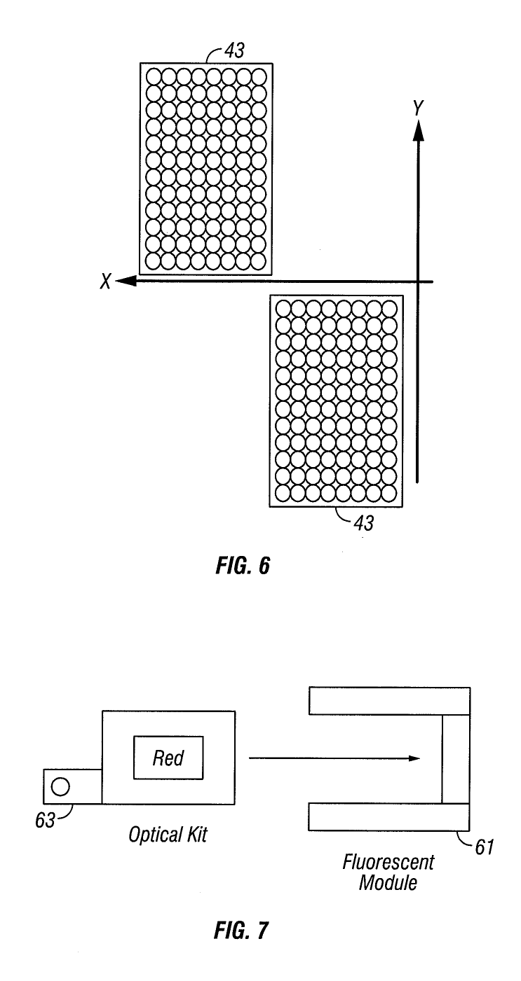

FIG. 6 is a block diagram showing microplate movement in a multi-mode modular apparatus for micro-titer analysis according to the invention. In the presently preferred embodiment of the invention, the plate moves in a Y direction first; the plate moves in an X direction second under the installed module. Microplate reading is performed well by well in this embodiment of the invention. Thus, a single platform may be used for any of several types of detection by providing common reader elements, such as a plate drive mechanism, in the platform, and adapting the platform to receive and be electronically and mechanically configured by a user installed detection module.

As discussed above, the invention receives any of a plurality of user installed detection modules. The individual modules are discussed below.

Fluorescence Module

The reader with the fluorescence module installed provides a high-performance fluorometer. The invention can be alternatively configured as a multimode reader for measuring fluorescence, luminescence, and absorbance by installing the luminescence and absorbance modules.

Superior performance is achieved by using a dedicated fluorescence detector instead of sharing the detector with other measurement modules. Optimum sensitivity is enabled with modern solid-state optics rather than traditional lamps and detectors. Each fluorescence optical kit (described below) employs a powerful LED to excite samples with energy at the optimum wavelength for the selected application. This results in superior sensitivity, specificity, and flexibility.

The fluorescence module features a dedicated optical design to read epifluorescence samples from above. A detection head and four fluorescence optical kits measure the most popular fluorophors (see Table 1 below). Optical kits can be easily exchanged in seconds. Instrument software ensures that the installed optical kit matches the selected application protocol.

Protocols for nucleic acids and proteins such as PicoGreen®, RiboGreen®, and Quant-iT™ assays are preprogrammed into the instrument for convenience. Cell-based fluorescence assays and gene expression assays using various fluorescent proteins can also be measured.

TABLE 1
Optical Kits

| Optical | Excitation | Emission | |
|---|---|---|---|
| Kit | Wavelength | Wavelength | Typical Fluorophors |
| UV | 365 nm | 410-460 nm | Hoechst dye, 4-MU |
| Blue | 460 nm | 515-580 nm | PicoGreen ®, RiboGreen ®, |
| | | | Fluorescein, Quant-iT ™ Protein, |
| | | | Quant-iT ™ dsDNA, EGFP, or |
| | | | rAcGFP |
| Green | 525 nm | 580-640 nm | Rhodamine, Cy ®3 |
| Red | 625 nm | 660-720 nm | Cy ®5, Quant-iT ™ RNA |

The following are typical specifications for a presently preferred embodiment of the invention:
Light Source: wavelength-matched LED
Detector: PIN-photodiode
Read Position: top reading
Wavelength Selection: snap-in Fluorescence Optical Kits
Wavelengths: UV (Ex: 365 nm, Em: 410-460 nm), Blue (Ex: 460 nm, Em: 515-580 nm), Green (Ex: 525 nm, Em: 580-640 nm), Red (Ex: 625 nm, Em: 660-720 nm)
Detection Limit: 0.5 fmol/200 μl or 1 ppt of fluorescein in 96-well plate
Linear Dynamic Range: 6 decades, assay dependent

FIG. 7 is a high level block diagram showing a fluorescence module 61 which mates with a corresponding channel 23 in the platform of a multi-mode modular apparatus for micro-titer analysis according to the invention. The preferred module comprises two components: the module 61 itself, which attaches to the instrument, and an optical kit 63, which snaps in and out of the module, thus allowing the user to change between applications quickly.

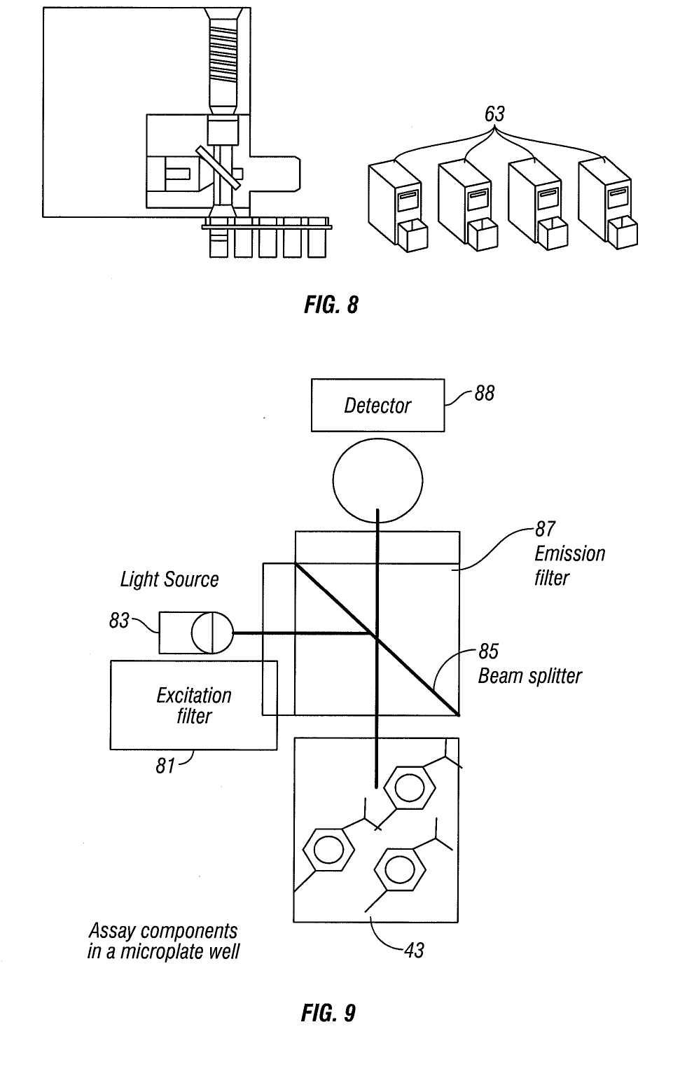

FIG. 8 is a more detailed block diagram showing a fluorescence module in a multi-mode modular apparatus for micro-titer analysis according to the invention. In FIG. 8, a plurality of optical kits 63 are shown, one for each of red, UV, blue, and green. Those skilled in the art will appreciate that other optical kits can be provided in connection with the invention. The optical kits configure the fluorescence module for detection of corresponding compounds, as shown on FIG. 8. Thus, the optical kits allow a user to configure a fluorescence module which, in turn, is used to configure the reader platform.

FIG. 9 is a schematic diagram showing a fluorescence module in a multi-mode modular apparatus for micro-titer analysis according to the invention. The module uses an epifluorescence setup. In such arrangement, light from a light source 83 passes through an excitation filter 81. The light is reflected into the sample 43 via a beam splitter 85. The molecules of interest are excited. Emission light is discriminated back through the beam splitter and passes through an emission filter 87 and into a detector 88. This arrangement allows for a low sample volume.

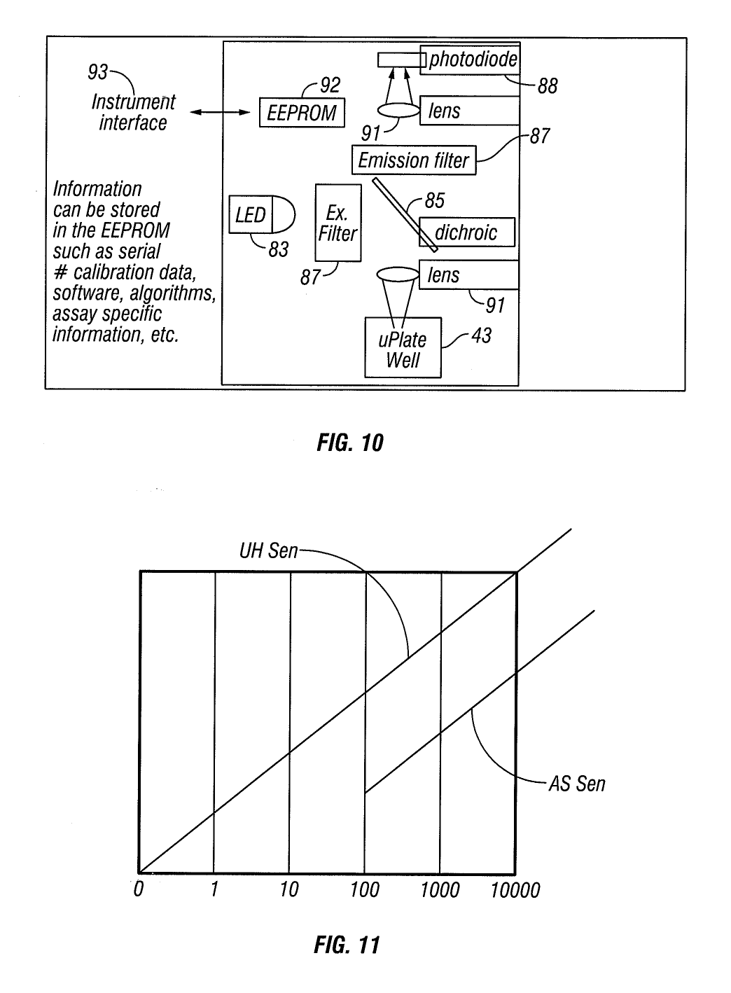

FIG. 10 is a block diagram showing a fluorescence module optical system in a multi-mode modular apparatus for micro-titer analysis according to the invention. Fluorescence detection is accomplished in this embodiment with an epi-fluorescent setup. A lens 91 is situated over the microplate well, and it both delivers light from an LED to the microplate and collects fluorescence from the microplate to a photodiode. FIG. 10 also shows an EEPROM 92, in which a module personality is stored. The EEPROM contains instructions that configure the platform, such that the reader is configured to operate as a fluorescence detector. The module is electronically coupled to the platform via an instrument interface 93 within the channel described above. The EEPROM stores such information as an instrument serial number, calibration information for calibrating the platform, software, processing algorithms for processing detection data, assay specific information, and the like. Those skilled in the art will appreciate that other storage means may be used in place of an EEPROM. A similar arrangement is used by each module to personalize the platform to perform the modules specific function.

Luminescence Module

The reader with the luminescence UHS module installed stands alone as a high-performance luminometer. The system can be alternatively configured as a multimode reader for measuring luminescence, fluorescence, and absorbance by installing the fluorescence and absorbance modules.

Superior performance is achieved by using a dedicated luminescence detector instead of sharing the detector with other measurement modules. Optimum sensitivity is achieved with low-noise circuitry, unique optical design, and a premium photon-counting photomultiplier tube (PMT). Minimal crosstalk is realized with dual-masking systems, where one mask covers the well while another covers the detector. Protocols for popular assays, such as Promega's Dual-Luciferase™ are preprogrammed into the reader for convenience.

The luminescence light plate provides an external control to ensure the luminometer is functioning properly. Some labs require this additional verification procedure. Reading the light plate before taking measurements is a quick and easy way to ensure quality control over linearity and consistency of readings.

The following are typical specifications for a presently preferred embodiment of the invention:
Detector: head-on photomultiplier tube (PMT) for photon counting
Wavelengths: 350-650 nm
Detection Limit: 3×10⁻²¹ moles of luciferase
Linear Dynamic Range: >8 decades
Cross-Talk: 5×10⁻⁶ using Costar #3789 plates

FIG. 11 is a graph showing luminescence sensitivity in a multi-mode modular apparatus for micro-titer analysis according to the invention. The presently preferred embodiment comprises two luminescent modules, i.e. an ultra high sensitivity (1×10−²⁰ moles luciferase using BrightGlo) module for detecting gene expression using luciferase, and a module for assay specific sensitivity (1×10−¹⁸ moles luciferase using BrightGlo). FIG. 11 shows a sensitivity curve for each of these modules. As can be seen, the modules provide broad dynamic range. In the preferred embodiment, both modules exhibit at least 8+ logs of dynamic range.

The luminescence module is photodiode-based, and uses a high-sensitivity photodiode/integrating op-amp circuit similar to that used in the GloRunner. The luminescence module is Photo-Multiplier Tube (PMT) based and can be either service-center or factory-installed onto the detector head translation stage, and is located so that users can still install and use the other modular heads. In other embodiments, the module is user installed. To achieve the desired crosstalk specification, the user is provided with an opaque microplate cover with 96 holes over the wells. As with other modules, information can be stored in an EEPROM (or any other memory device), such as serial number, calibration data, which addresses or/and enables proper software, algorithms, assay specific information, and the like.

Absorbance Module

The following are typical specifications for a presently preferred embodiment of the invention:
Light Source: LED
Detector: large-area photodiode
Spectral Range: 360-800 nm
Filter Wheel Capacity: holds up to six filters. Includes four installed filters and two empty filter holders for user configuration
Wavelengths for Installed Filters: 450, 550, 600, 750 nm
Photometric Measuring Range: 0-4.0 OD
Linear Dynamic Range: 0-2.5 OD
OD Accuracy: 0.01 OD ±3% 2.5 OD
OD Precision: 0.01 OD ±1%

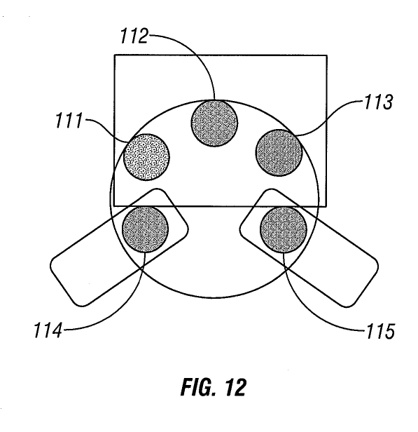

FIG. 12 is a block diagram showing an absorbance module in a multi-mode modular apparatus for micro-titer analysis according to the invention. The absorbance module can be either user or factory installed. The presently preferred embodiment provides five filter positions, comprising three fixed filters 11, 112, 113 and two variable filters 114, 115. The user can select ratiometric measurements for two compound absorbance.

Absorbance filter wavelengths include: 550 nm, 600 nm, 750 nm, and 405 nm, although other wavelengths may be provided as appropriate.

The absorbance head is supplied with multiple filters in a motor-driven cartridge or wheel. This cartridge has room for two additional filters that can be installed by the user. The filter positions are recognizable by the instrument, and are controlled by the user interface (discussed below). As with the other user installable module, information can be stored in an EEPROM, such as serial number, calibration data, software, algorithms, assay specific information, and the like.

Pump Module

The pump module holds one or two injector pumps. It can be a very simple industrial design, sitting on top of or behind the instrument. The tubing should be as short as possible to minimize dead volume and it is important to guard against light-piping of ambient light into the detection region. The injectors are mounted on the detector head translation stage. Injectors are available for all modules, even if a luminometer module is not present. As with other modules, information can be stored in an EEPROM, such as serial number, calibration data, software, algorithms, assay specific information, ands the like.

Software

Embedded software, developed on a Windows CE platform, is embedded in the reader. The software allows complete control of the reader to be performed from the touch screen without a connection to a PC. For data output, the reader can write to a thumb-drive type memory device, which interfaces to the reader through a USB port at the front of the instrument. The data files it produces are readable by a computer running Windows XP or above.

Embedded GUI software and low level software updates can be accomplished in one of two ways:

Over USB: A client connection is provided on the back of the instrument. In this case, the user can download the updated embedded software from their PC to the Instrument.

Over the USB-host connection on the front of the reader: In this case, the user can attach a thumb-drive with updated firmware to the reader, and press a menu-item on the touch screen to initiate software upgrade.

In both cases, the user can get the updated firmware either by downloading it off a website, or from a CD/DVD.

User Interface

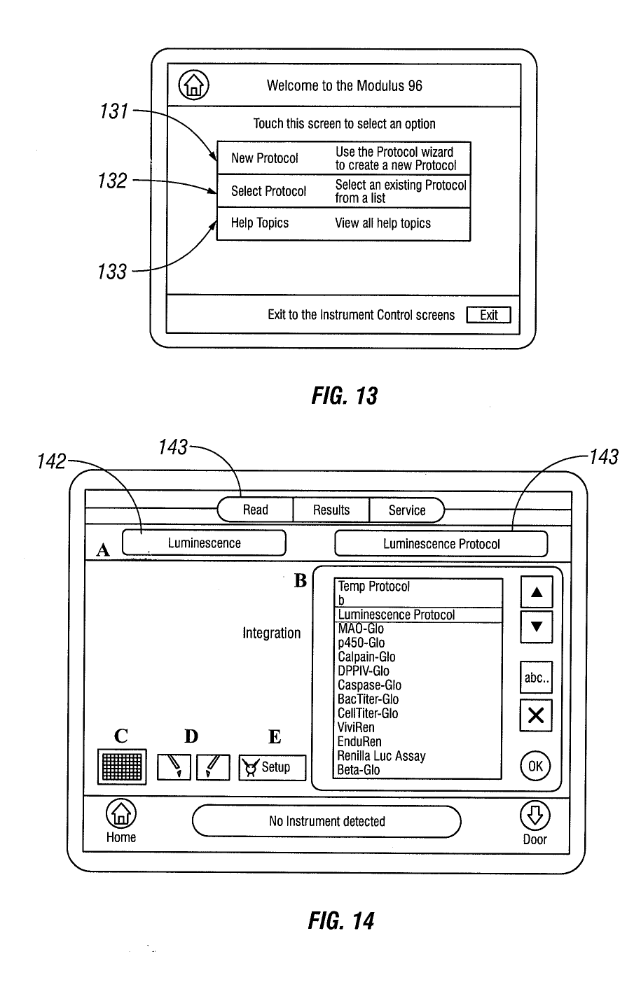

FIG. 13 is a screen shot of a user interface in a multi-mode modular apparatus for micro-titer analysis according to the invention. The user interface provides a protocol wizard 131 that walks user through step-by-step setup of protocols; user access to stored protocols 132, such as Promega protocols-luminescence, Invitrogen protocols-fluorescence, and various other selectable protocols; and online help 133, by which a user can view help topics and get context sensitive help.

User Interface-Read Screen

FIG. 14 is a screen shot of a user interface read screen in a multi-mode modular apparatus for micro-titer analysis according to the invention. When the read button 141 is selected, a read page is displayed. A user may touch the Luminescence bar 142 to toggle between technologies. The user may select protocols from a list 143, set up well selection, view parameters, and use an injector wizard.

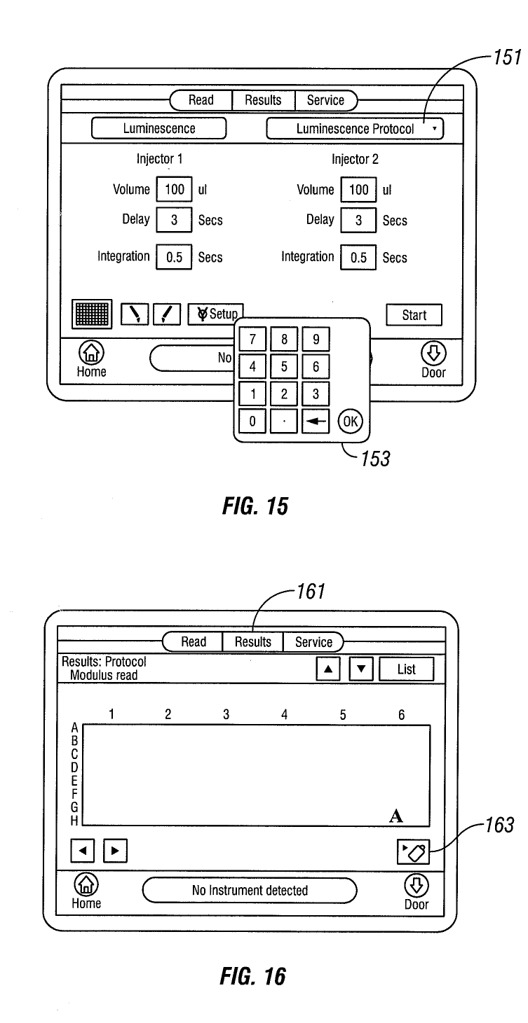

FIG. 15 is a screen shot of a user interface read screen showing setup and modification of protocols in a multi-mode modular apparatus for micro-titer analysis according to the invention. In FIG. 16, the user has selected a Luminescence Protocol bar 151 to setup or modify protocols. and touch parameters. A user numeric keypad 153 allows the user to enter values.

User Interface-Results Screen

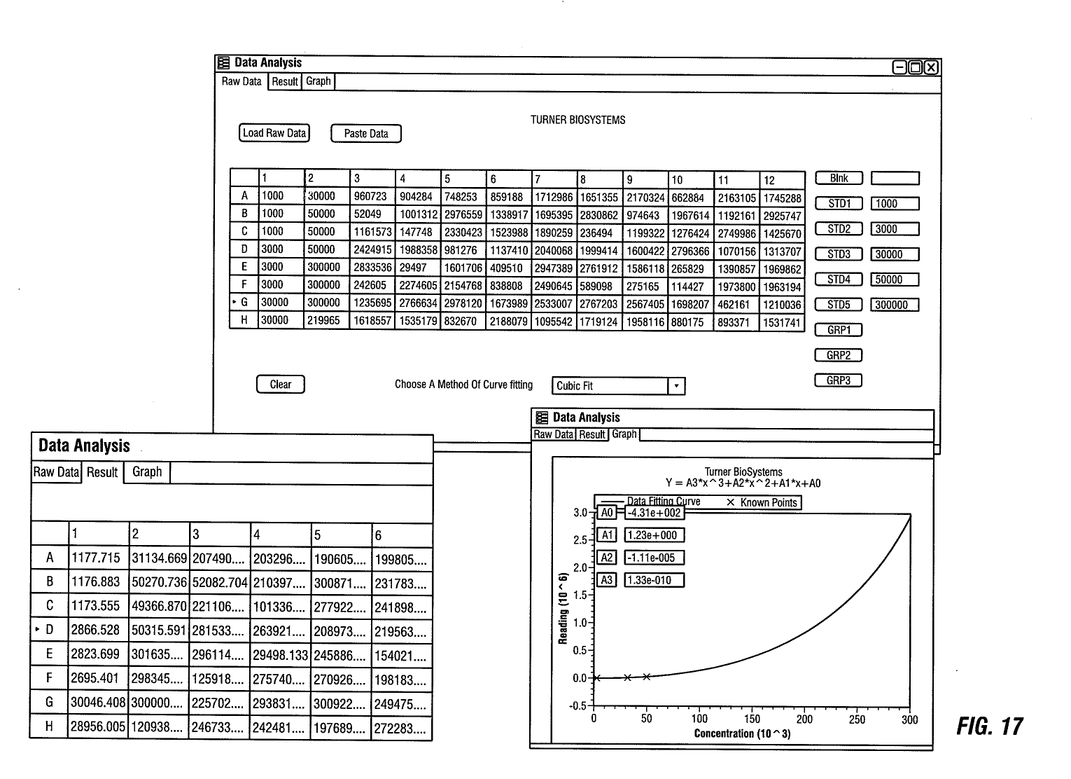

FIG. 17 is a screen shot of a user interface results screen in a multi-mode modular apparatus for micro-titer analysis according to the invention. When the Results bar 161 is selected, results are presented on the screen and are maximized for easy viewing by the user. The user may touch a thumb stick icon 163 to send data to the thumb stick via the USB port on the front of the reader, or the user may elect to store data on the reader and send it to thumb stick in batch form.

User Interface-Additional Data Handling

FIG. 17 is a screen shot of a user interface read screen showing additional data handling in a multi-mode modular apparatus for micro-titer analysis according to the invention. A curve fitting program allow a user to paste data from an Excel spreadsheet, e.g. by loading the data from a thumb stick. The user sets appropriate standards, selects a curve fit, and clicks a button for a result. Curve fitting data analysis software enables concentration calculation, graphing, and printing. In the preferred embodiment, eight different curve-fitting methods are available: linear fit, quadratic fit, cubic fit, two-parameter fit, four-parameter with linear x-axis fit, four-parameter with log two-axis fit, point-to-point, and cubic spline. This software is preferably compatible with the Windows XP operating system for PC computers.

Although the invention is described herein with reference to the preferred embodiment, one skilled in the art will readily appreciate that other applications may be substituted for those set forth herein without departing from the spirit and scope of the present invention. Accordingly, the invention should only be limited by the Claims included below.

1. A reconfigurable microplate reader, comprising:
a plurality of user installable detection modules, each module comprising a self-contained detector for a microplate reader, each module reconfiguring said microplate reader to implement at least one specific detection scheme for detecting any of luminescence, fluorescence, and absorbance and/or reconfiguring said microplate reader to implement any of a fluorometer and a luminometer; and
a microplate reader platform comprising a port for coupling at least one user installable detection module thereto, said platform further comprising a user interface from said platform to said module, a machine interface from said platform to said module, and a platform configuration mechanism for recognizing a specific detection scheme for an installed detection module and for configuring said platform to support said specific detection scheme.

2. The reconfigurable microplate reader of claim 1, wherein said microplate reader is upgradable or reconfigurable by a user from an initial configuration having an initial set of detection modalities and other features, as received, to a new configuration having a second set of detection modalities and other features, s to accommodate a different set of biological and biochemical assays without requiring a field service visit or return for upgrading.

3. The reconfigurable microplate reader of claim 1, said platform further comprising:
an interface for any of external or internal control or programming; data transfer, optionally using transportable memory devices; data analysis; functional enhancements, including addition of any of bar code readers and RF ID tags; Ethernet; and other network interfaces.

4. The reconfigurable microplate reader of claim 1, said user interface further comprising:
a touch screen display.

5. The reconfigurable microplate reader of claim 1, further comprising:
meas for user upgradeability and data transportability via memory devices.

6. The reconfigurable microplate reader of claim 1, said memory devices further comprising:
a USB drive.

7. A multi-mode modular apparatus for micro-titer analysis, comprising:
at least one user installable module; and
a platform adapted to receive said at least one user installable module to configure said apparatus to function as any of a luminescence detector in either of two or more modes, a fluorescence detector, an absorbance detector, a dedicated fluorometer, and a dedicated luminometer.

8. The apparatus of claim 1, further comprising:
a plurality of dedicated channels for detection of different parameters, where each parameter detected comprises a specific position on a rack within said platform.

9. The apparatus of claim 8, said channels further comprising:
three specific positions, one each for absorbance, fluorescence, and luminescence high sensitivity/luminescence assay specific.

10. The apparatus of claim 7, wherein each module can be installed or removed separately.

11. The apparatus of claim 8, wherein each channel is dedicated.

12. The apparatus of claim 8, wherein each channel is electrically defined by a series of pins in a connector within a single bay in said platform, wherein said platform is configurable to support a single detector at any given time.

13. The apparatus of claim 7, further comprising:
a separate, dedicated bay for each module.

14. The apparatus of claim 7, said platform comprising any of:
a light source for use in absorbance detection;
an XY plate stage for effecting XY movement of a microplate; and
an external pump module; and an external power source for said pump module coupled to said platform for use when said apparatus is configured for absorbance detection.

15. The apparatus of claim 7, further comprising:
a low level microprocessor for controlling said at least one module; and
a high level microprocessor for controlling said platform is controlled.

16. The apparatus of claim 15, wherein said high level microprocessor supports a user interface comprising a touch screen and user accessible USB connector which allows a user to load and store data to and from said apparatus with an external USB drive.

17. The apparatus of claim 7, said at least one module comprising:
a fluorescence module having a dedicated optical design for reading epifluorescence samples from above, and comprising a detection head, a plurality of fluorescence optical kits for measuring fluorophors, and instrument software for ensuring that an installed optical kit matches a selected application protocol;
wherein protocols for any of nucleic acid assays, protein assays, cell-based fluorescence assays, and gene expression assays are preprogrammed into said module.

18. The apparatus of claim 7, further said at least one module further comprising:
a memory containing instructions for configuring said platform to operate as a specific detector when said module is electronically coupled to said platform via an instrument interface within a channel;
wherein said memory stores information comprising any of an instrument serial number, calibration information for calibrating the platform, software, processing algorithms for processing detection data, and assay specific information.

19. The apparatus of claim 7, said at least one module comprising:
a luminescence module comprising a dedicated luminescence detector, said dedicated luminescence detector comprising any of a high sensitivity photodiode and a photon-counting photomultiplier tube (PMT); and
a dual-masking system, in which one mask covers a well while another mask covers said detector;
wherein protocols for assays are preprogrammed into said module;
said module comprising any of an ultra high sensitivity (1×10−²⁰ moles luciferase using BrightGlo) module for detecting gene expression using luciferase, and a module for assay specific sensitivity (1×10−¹⁸ moles luciferase using BrightGlo).

20. The apparatus of claim 7, said at least one module comprising:
an absorbance module comprising a plurality of filter positions, comprising any of fixed filters and variable filters, in a motor-driven cartridge or wheel;
wherein filter positions are recognizable by said module and are controlled by a user interface.

21. The apparatus of claim 7, said at least one module comprising:
a pump module holding one or more injector pumps;
a plurality of injectors mounted on a detector head translation stage.

22. A method for reconfiguring a microplate reader, comprising the steps of:
providing a plurality of user installable detection modules, each module comprising a self-contained detector for a microplate reader, each module reconfiguring said microplate reader to implement at least one specific detection scheme for detecting any of luminescence, fluorescence, and absorbance and/or reconfiguring said microplate reader to implement any of a fluorometer and a luminometer;
providing a microplate reader platform comprising a port for coupling at least one user installable detection module thereto, said platform further comprising a user interface from said platform to said module, a machine interface from said platform to said module, and a platform configuration mechanism for recognizing a specific detection scheme for an installed detection module and for configuring said platform to support said specific detection scheme; and
installing at least one user installable detection module into said platform.
